(19) United States
(12) Patent Application Publication
(10) Pub. No.: US 2024/0033365 A1
(43) Pub. Date: Feb. 1, 2024

(54) POLY(2-OXAZOLINE)N-f-MLF DERIVATIVE CONJUGATES THAT INHIBIT FORMYL PEPTIDE RECEPTORS FOR THE TREATMENT OF DISEASE

(71) Applicant: University of South Alabama, Mobile, AL (US)

(72) Inventors: Andrew E. Byrd, Mobile, AL (US); Robert A. Barrington, Mobile, AL (US)

(73) Assignee: University of South Alabama, Mobile, AL (US)

(21) Appl. No.: 18/224,879

(22) Filed: Jul. 21, 2023

Related U.S. Application Data
(60) Provisional application No. 63/391,378, filed on Jul. 22, 2022.

(51) Int. Cl. A61K 47/64; A61K 45/06

(52) U.S. Cl. CPC A61K 47/64; A61K 45/06

(57) ABSTRACT

Described are the compounds, compositions and methods of treating diseases and disorders characterized by Formyl Peptide Receptor 1 (FPR1) or FPR2 activity.

### RELATED APPLICATIONS

This application claims the benefit of priority under 35 U.S.C. § 119(e) to U.S. Provisional Application No: 63/391,378, filed Jul. 22, 2022, which is incorporated herein by reference in its entirety.

### BACKGROUND

Formyl peptide receptors (FPRs) are G protein-coupled receptors. Their main function is to sense the presence of harmful or noxious molecules such as formylated peptides and guide cells to the site where pathogen-associated molecules have been released (Ye, et al., Pharmacol. Rev. 61:119-161 (2009)). This sensing function of FPRs is not limited to a particular pathogen. It extends to a wide range of endogenous ligands including classical biomarkers of inflammation and immune activation such as serum amyloid A (SAA) (Su, et al., J. Exp. Med. 189:395-402 (1999)), formylated peptides released by mithochondria of damaged cells and tissue (Zhang, et al., Nature 464:104-107 (2010)), the antimicrobial peptide LL-37 (De, et al., J. Exp. Med. 192:1069-1074 (2000)) and the dual pro- and anti-inflammatory protein Annexin-A1.

There are three known functional FPRs in humans, FPR1, FPR2 and FPR3. Each recognizes, to different degrees, a wide range of endogenous and exogenous ligands (Panaro, et al., Immunopharmacol. Immunotoxicol. 28:103-127 (2006); Dufton, et al., Pharmacol. Ther. 127:175-188 (2010)). Activation of these receptors causes their homo- or hetero-dimerization which in turn depends on the precise ligand to which they bind (Cooray, et al., Proc. Natl. Acad. Sci. USA 110:18232-18237 (2013)). In this way FPRs are able to exert both pro- and anti-inflammatory effects on immune cells. The expression of FPRs is highest in sentinel innate cells with phagocytic or chemotactic activity such as neutrophils (Spurr, et al., Int. Immunopharmacol. 11:55-66 (2011)), monocytes (Yang, et al., J. Immunol. 166:4092-4098 (2001), macrophages (Gemperle, et al., PLos One 7:e50195 (2012)) and dendritic cells (Yang, et al., J. Leukoc. Biol. 72:598-607 (2002)). However, FPRs are also expressed in non-phagocytic and “immobile” sentinel cells such as mucosal epithelial cells (Babbin, et al., J. Immunol. 179:8112-8121 (2007)), endothelial cells (Heo, et al., Stem Cells 32:779-790 (2014)) and glia (Chen, et al., J. Immunol. 178:1759-1766 (2007)).

Binding of antigen to FPR leads to myeloid cell migration, inflammatory-mediator release, increased phagocytosis and gene transcription (Chen, et al., J. Autoimmun. 85:64-77 (2017); Dorward, et al., Am. J. Pathol. 185:1172-1184 (2015)). Manipulation of FPRs with modified peptides can inhibit inflammatory effects induced by IgE binding to an antigen, having potential anti-myeloid cell migration effects, including lung infiltration in asthma.

### SUMMARY

A first aspect of the present disclosure is directed to a compound represented by Formula I:

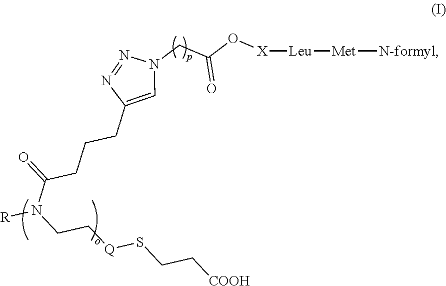

wherein o, p, Q, R and X are as defined herein, or a pharmaceutically acceptable salt or stereoisomer.

In another aspect, pharmaceutical compositions containing a therapeutically effective amount of the compound of Formula (I), or a pharmaceutically acceptable salt or stereoisomer thereof, and a pharmaceutically acceptable carrier are provided.

Yet another aspect of the present disclosure is directed to method of treating a disease or disorder mediated by Formyl Peptide Receptor (FPR) 1 or 2 (hereinafter “FPR1 and 2” or “FPR1 and FPR2”), comprising administering to a subject in need thereof a therapeutically effective amount of a compound of Formula (I), or a pharmaceutically acceptable salt or stereoisomer thereof.

In some embodiments of the disclosed methods, the disease or disorder is an inflammatory disease or disorder.

In some embodiments, the inflammatory disease or order is characterized by mucosal inflammation. In some embodiments, the inflammatory disease or disorder is a respiratory disease or disorder, asthma, chronic obstructive pulmonary disease (COPD), eczema, acute lung injury, arthritis, psoriasis, or an intestinal inflammatory disease or disorder. In some embodiments, the intestinal inflammatory disease or disorder is colitis. In some embodiments, the disease or disorder is a hyper-IgE syndrome (HIES). In some embodiments, the HIES is autosomal dominant hyper-IgE syndrome (AD-HIES).

### DETAILED DESCRIPTION

Unless defined otherwise, all technical and scientific terms used herein have the same meaning as is commonly understood by one of skill in the art to which the subject matter herein belongs. As used in the specification and the appended claims, unless specified to the contrary, the following terms have the meanings indicated in order to facilitate the understanding of the present disclosure.

As used in the description and the appended claims, the singular forms “a”, “an”, and “the” include plural referents unless the context clearly dictates otherwise. Thus, for example, reference to “a composition” includes mixtures of two or more such compositions, reference to “an inhibitor” includes mixtures of two or more such inhibitors, and the like.

Unless specifically stated or obvious from context, as used herein, the term “about” is understood as within a range of normal tolerance in the art, for example within 2 standard deviations of the mean. “About” can be understood as within 10%, 9%, 8%, 7%, 6%, 5%, 4%, 3%, 2%, 1%, 0.5%, 0.1%, 0.05%, or 0.01% of the stated value. Unless otherwise clear from context, all numerical values provided herein are modified by the term “about”.

Ranges can be expressed herein as from “about” one particular value and/or to “about” another particular value. When such a range is expressed, another aspect includes from the one particular value and/or to the other particular value. Similarly, when values are expressed as approximations, by use of the antecedent “about,” it is understood that the particular value forms another aspect. It is further understood that the endpoints of each of the ranges are significant both in relation to the other endpoint, and independently of the other endpoint. It is also understood that there are a number of values disclosed herein, and that each value is also herein disclosed as “about” that particular value in addition to the value itself. It is also understood that throughout the application, data are provided in a number of different formats and that this data represent endpoints and starting points and ranges for any combination of the data points. For example, if a particular data point “10” and a particular data point “15” are disclosed, it is understood that greater than, greater than or equal to, less than, less than or equal to, and equal to 10 and 15 are considered disclosed as well as between 10 and 15. It is also understood that each unit between two particular units are also disclosed. For example, if 10 and 15 are disclosed, then 11, 12, 13, and 14 are also disclosed. In this regard, ranges provided herein are understood to be shorthand for all of the values within the range. For example, a range of 1 to 50 is understood to include any number, combination of numbers, or sub-range from the group consisting 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 as well as all intervening decimal values between the aforementioned integers such as, for example, 1.1, 1.2, 1.3, 1.4, 1.5, 1.6, 1.7, 1.8, and 1.9. With respect to sub-ranges, “nested sub-ranges” that extend from either end point of the range are specifically contemplated. For example, a nested sub-range of an exemplary range of 1 to 50 may comprise 1 to 10, 1 to 20, 1 to 30, and 1 to 40 in one direction, or 50 to 40, 50 to 30, 50 to 20, and 50 to 10 in the other direction.

The transitional term “comprising,” which is synonymous with “including,” “containing,” or “characterized by,” is inclusive or open-ended and does not exclude additional, unrecited elements or method steps. When used in the context of the number of heteroatoms in a heterocyclic structure, it means that the heterocyclic group that that minimum number of heteroatoms. By contrast, the transitional phrase “consisting of” excludes any element, step, or ingredient not specified in the claim. The transitional phrase “consisting essentially of” limits the scope of a claim to the specified materials or steps “and those that do not materially affect the basic and novel characteristic(s)” of the disclosure.

With respect to compounds of the present disclosure, and to the extent the following terms are used herein to further describe them, the following definitions apply.

As used herein, the term “alkyl” refers to a saturated linear or branched-chain monovalent hydrocarbon radical. In one embodiment, the alkyl radical is a C₁-C₁₈ group. In some embodiments, and to the extent not disclosed otherwise for any one or more groups of the compounds of formula (I), the alkyl radical is a C₀-C₆, C₀-C₅, C₀-C₃, C₁-C₁₂, C₁-C₈, C₁-C₆, C₁-C₅, C₁-C₄ or C₁-C₃ group (wherein C₀ alkyl refers to a bond). Examples of alkyl groups include methyl, ethyl, 1-propyl, 2-propyl, i-propyl, 1-butyl, 2-methyl-1-propyl, 2-butyl, 2-methyl-2-propyl, 1-pentyl, n-pentyl, 2-pentyl, 3-pentyl, 2-methyl-2-butyl, 3-methyl-2-butyl, 3-methyl-1-butyl, 2-methyl-1-butyl, 1-hexyl, 2-hexyl, 3-hexyl, 2-methyl-2-pentyl, 3-methyl-2-pentyl, 4-methyl-2-pentyl, 3 -methyl -3 -pentyl, 2-methyl-3 -pentyl, 2,3 -dimethyl-2-butyl, 3,3 -dimethyl-2-butyl, heptyl, octyl, nonyl, decyl, undecyl and dodecyl. In some embodiments, an alkyl group is a C₁-C₃ alkyl group. In some embodiments, an alkyl group is a C₁-C₂ alkyl group, or a methyl group.

As used herein, the term “alkenyl” refers to a linear or branched-chain monovalent hydrocarbon radical with at least one carbon-carbon double bond. An alkenyl includes radicals having “cis” and “trans” orientations, or alternatively, “E” and “Z” orientations. In one example, the alkenyl radical is a C₂-C₁₈ group. In some embodiments, and to the extent not disclosed otherwise for any one or more groups of the compounds of formula (I), the alkenyl radical is a C₂-C₁₂, C₂-C₁₀, C₂-C₈, C₂-C₆, or C₂-C₃group. Examples include ethenyl or vinyl, prop-1-enyl, prop-2-enyl, 2-methylprop-1-enyl, but-1-enyl, but-2-enyl, but-3-enyl, buta-1,3-dienyl, 2-methylbuta-1,3-diene, hex-1-enyl, hex-2-enyl, hex-3-enyl, hex-4-enyl and hexa-1,3-dienyl.

As used herein, the term “alkynyl” refers to a linear or branched monovalent hydrocarbon radical with at least one carbon-carbon triple bond. In one example, the alkynyl radical is a C₂-C₁₈ group. In some embodiments, and to the extent not disclosed otherwise for any one or more groups of the compounds of formula (I), the alkynyl radical is C₂-C₁₂, C₂-C₁₀, C₂-C₈, C₂-C₆, or C₂-C₃. Examples include ethynyl prop-1-ynyl, prop-2-ynyl, but-1-ynyl, but-2-ynyl and but-3-ynyl.

The terms “alkoxyl” or “alkoxy” as used herein refer to an alkyl group, as defined above, having an oxygen radical attached thereto, and which is the point of attachment. Representative alkoxyl groups include methoxy, ethoxy, propyloxy, tert-butoxy and the like. An “ether” is two hydrocarbyl groups covalently linked by an oxygen. Accordingly, the substituent of an alkyl that renders that alkyl an ether is or resembles an alkoxyl, such as can be represented by one of -O-alkyl, -O-alkenyl, and -O-alkynyl.

As used herein, the term “halogen” (or “halo” or “halide”) refers to fluorine, chlorine, bromine, or iodine.

As used herein, the term “cyclic group” broadly refers to any group that used alone or as part of a larger moiety, contains a saturated, partially saturated or aromatic ring system e.g., carbocyclic (cycloalkyl, cycloalkenyl), heterocyclic (heterocycloalkyl, heterocycloalkenyl), aryl and heteroaryl groups. Cyclic groups may have one or more (e.g., fused) ring systems. Thus, for example, a cyclic group can contain one or more carbocyclic, heterocyclic, aryl or heteroaryl groups.

As used herein, the term “carbocyclic” (also “carbocyclyl”) refers to a group that used alone or as part of a larger moiety, contains a saturated, partially unsaturated, or aromatic ring system having 3 to 20 carbon atoms, that is alone or part of a larger moiety (e.g., an alkcarbocyclic group). The term carbocyclyl includes mono-, bi-, tri-, fused, bridged, and spiro-ring systems, and combinations thereof. In one embodiment, carbocyclyl includes 3 to 15 carbon atoms (C₃-C₁₅). In one embodiment, carbocyclyl includes 3 to 12 carbon atoms (C₃-C₁₂). In some embodiments, and to the extent not disclosed otherwise for any one or more groups of the compounds of formula (I), carbocyclyl includes C₃-C₈, C₃-C₁₀ or C₅-C₁₀. In another embodiment, carbocyclyl, as a monocycle, includes C₃-C₈,C₃-C₆ or C₅-C₆. In some embodiments, carbocyclyl, as a bicycle, includes C₇-C₁₂. In another embodiment, carbocyclyl, as a spiro system, includes C₅-C₁₂. Representative examples of monocyclic carbocyclyls include cyclopropyl, cyclobutyl, cyclopentyl, 1-cyclopent-1-enyl, 1-cyclopent-2-enyl, 1-cyclopent-3-enyl, cyclohexyl, perdeuteriocyclohexyl, 1-cyclohex-1-enyl, 1-cyclohex-2-enyl, 1-cyclohex-3-enyl, cyclohexadienyl, cycloheptyl, cyclooctyl, cyclononyl, cyclodecyl, cycloundecyl, phenyl, and cyclododecyl; bicyclic carbocyclyls having 7 to 12 ring atoms include [4,3], [4,4], [4,5], [5,5], [5,6] or [6,6] ring systems, such as for example bicyclo[2.2.1]heptane, bicyclo[2.2.2]octane, naphthalene, and bicyclo[3.2.2]nonane. Representative examples of spiro carbocyclyls include spiro[2.2]pentane, spiro[2.3]hexane, spiro[2.4]heptane, spiro[2.5]octane and spiro[4.5]decane. The term carbocyclyl includes aryl ring systems as defined herein. The term carbocycyl also includes cycloalkyl rings (e.g., saturated or partially unsaturated mono-, bi-, or spiro-carbocycles). The term carbocyclic group also includes a carbocyclic ring fused to one or more (e.g., 1, 2 or 3) different cyclic groups (e.g., aryl or heterocyclic rings), where the radical or point of attachment is on the carbocyclic ring.

Thus, the term carbocyclic also embraces carbocyclylalkyl groups which as used herein refer to a group of the formula —R^{c}-carbocyclyl where R^{c} is an alkylene chain. The term carbocyclic also embraces carbocyclylalkoxy groups which as used herein refer to a group bonded through an oxygen atom of the formula —O—R^{c}-carbocyclyl where R^{c} is an alkylene chain.

As used herein, the term “aryl” used alone or as part of a larger moiety (e.g., “aralkyl”, wherein the terminal carbon atom on the alkyl group is the point of attachment, e.g., a benzyl group),“aralkoxy” wherein the oxygen atom is the point of attachment, or “aroxyalkyl” wherein the point of attachment is on the aryl group) refers to a group that includes monocyclic, bicyclic or tricyclic, carbon ring system, that includes fused rings, wherein at least one ring in the system is aromatic. In some embodiments, the aralkoxy group is a benzoxy group. The term “aryl” may be used interchangeably with the term “aryl ring”. In one embodiment, aryl includes groups having 6-18 carbon atoms. In another embodiment, aryl includes groups having 6-10 carbon atoms. Examples of aryl groups include phenyl, naphthyl, anthracyl, biphenyl, phenanthrenyl, naphthacenyl, 1,2,3,4-tetrahy dronaphthalenyl, 1H-indenyl, 2,3 -dihydro-1H-indenyl, naphthyridinyl, and the like, which may be substituted or independently substituted by one or more substituents described herein. A particular aryl is phenyl. In some embodiments, an aryl group includes an aryl ring fused to one or more (e.g., 1, 2 or 3) different cyclic groups (e.g., carbocyclic rings or heterocyclic rings), where the radical or point of attachment is on the aryl ring.

Thus, the term aryl embraces aralkyl groups (e.g., benzyl) which as disclosed above refer to a group of the formula —R^{c}-aryl where R^{c} is an alkylene chain such as methylene or ethylene. In some embodiments, the aralkyl group is an optionally substituted benzyl group. The term aryl also embraces aralkoxy groups which as used herein refer to a group bonded through an oxygen atom of the formula —O—R^{c}-aryl where R^{c} is an alkylene chain such as methylene or ethylene.

As used herein, the term “heterocyclyl” refers to a “carbocyclyl” that used alone or as part of a larger moiety, contains a saturated, partially unsaturated or aromatic ring system, wherein one or more (e.g., 1, 2, 3, or 4) carbon atoms have been replaced with a heteroatom (e.g., O, N, N(O), S, S(O), or S(O)₂). The term heterocyclyl includes mono-, bi-, tri-, fused, bridged, and spiro-ring systems, and combinations thereof. In some embodiments, a heterocyclyl refers to a 3 to 15 membered heterocyclyl ring system. In some embodiments, a heterocyclyl refers to a 3 to 12 membered heterocyclyl ring system. In some embodiments, a heterocyclyl refers to a saturated ring system, such as a 3 to 12 membered saturated heterocyclyl ring system. In some embodiments, a heterocyclyl refers to a heteroaryl ring system, such as a 5 to 14 membered heteroaryl ring system. The term heterocyclyl also includes C₃-C₈ heterocycloalkyl, which is a saturated or partially unsaturated mono-, bi-, or spiro-ring system containing 3-8 carbons and one or more (1, 2, 3 or 4) heteroatoms.

In some embodiments, a heterocyclyl group includes 3-12 ring atoms and includes monocycles, bicycles, tricycles and spiro ring systems, wherein the ring atoms are carbon, and one to 5 ring atoms is a heteroatom such as nitrogen, sulfur or oxygen. In some embodiments, heterocyclyl includes 3- to 7-membered monocycles having one or more heteroatoms selected from nitrogen, sulfur or oxygen. In some embodiments, heterocyclyl includes 4- to 6-membered monocycles having one or more heteroatoms selected from nitrogen, sulfur or oxygen. In some embodiments, heterocyclyl includes 3-membered monocycles. In some embodiments, heterocyclyl includes 4-membered monocycles. In some embodiments, heterocyclyl includes 5-6 membered monocycles. In some embodiments, the heterocyclyl group includes 0 to 3 double bonds. In any of the foregoing embodiments, heterocyclyl includes 1, 2, 3 or 4 heteroatoms. Any nitrogen or sulfur heteroatom may optionally be oxidized (e.g., NO, SO, SO₂), and any nitrogen heteroatom may optionally be quaternized (e.g., [NR₄]⁺Cl⁻, [NR₄]⁺OH⁻). Representative examples of heterocyclyls include oxiranyl, aziridinyl, thiiranyl, azetidinyl, oxetanyl, thietanyl, 1,2-dithietanyl, 1,3-dithietanyl, pyrrolidinyl, dihydro-1H-pyrrolyl, dihydrofuranyl, tetrahydropyranyl, dihydrothienyl, tetrahydrothienyl, imidazolidinyl, piperidinyl, piperazinyl, morpholinyl, thiomorpholinyl, 1,1-dioxo-thiomorpholinyl, dihydropyranyl, tetrahydropyranyl, hexahydrothiopyranyl, hexahydropyrimidinyl, oxazinanyl, thiazinanyl, thioxanyl, homopiperazinyl, homopiperidinyl, azepanyl, oxepanyl, thiepanyl, oxazepinyl, oxazepanyl, diazepanyl, 1,4-diazepanyl, diazepinyl, thiazepinyl, thiazepanyl, tetrahydrothiopyranyl, oxazolidinyl, thiazolidinyl, isothiazolidinyl, 1,1-dioxoisothiazolidinonyl, oxazolidinonyl, imidazolidinonyl, 4,5,6,7-tetrahydro[2H]indazolyl, tetrahydrobenzoimidazolyl, 4,5,6,7-tetrahydrobenzo[d]imidazolyl, 1,6-dihydroimidazol[4,5-d]pyrrolo[2,3-b]pyridinyl, thiazinyl, thiophenyl, oxazinyl, thiadiazinyl, oxadiazinyl, dithiazinyl, dioxazinyl, oxathiazinyl, thiatriazinyl, oxatriazinyl, dithiadiazinyl, imidazolinyl, dihydropyrimidyl, tetrahydropyrimidyl, 1-pyrrolinyl, 2-pyrrolinyl, 3-pyrrolinyl, indolinyl, thiapyranyl, 2H-pyranyl, 4H-pyranyl, dioxanyl, 1,3-dioxolanyl, pyrazolinyl, pyrazolidinyl, dithianyl, dithiolanyl, pyrimidinonyl, pyrimidindionyl, pyrimidin-2,4-dionyl, piperazinonyl, piperazindionyl, pyrazolidinylimidazolinyl, 3-azabicyclo[3.1.0]hexanyl, 3,6-diazabicyclo[3.1.1]heptanyl, 6-azabicyclo[3.1.1]heptanyl, 3-azabicyclo[3.1.1]heptanyl, 3-azabicyclo[4.1.0]heptanyl, azabicyclo[2.2.2]hexanyl, 2-azabicyclo[3.2.1]octanyl, 8-azabicyclo[3.2.1]octanyl, 2-azabicyclo[2.2.2]octanyl, 8-azabicyclo[2.2.2]octanyl, 7-oxabicyclo[2.2.1]heptane, azaspiro[3.5]nonanyl, azaspiro[2.5]octanyl, azaspiro[4.5]decanyl, 1-azaspiro[4.5]decan-2-only, azaspiro[5.5]undecanyl, tetrahydroindolyl, octahydroindolyl, tetrahydroisoindolyl, tetrahydroindazolyl, 1,1-dioxohexahydrothiopyranyl. Examples of 5-membered heterocyclyls containing a sulfur or oxygen atom and one to three nitrogen atoms are thiazolyl, including thiazol-2-yl and thiazol-2-ylN-oxide, thiadiazolyl, including 1,3,4-thiadiazol-5-yl and 1,2,4-thiadiazol-5-yl, oxazolyl, for example oxazol-2-yl, and oxadiazolyl, such as 1,3,4-oxadiazol-5-yl, and 1,2,4-oxadiazol-5-yl. Example 5-membered ring heterocyclyls containing 2 to 4 nitrogen atoms include imidazolyl, such as imidazol-2-yl; triazolyl, such as 1,3,4-triazol-5-yl; 1,2,3-triazol-5-yl, 1,2,4-triazol-5-yl, and tetrazolyl, such as 1H-tetrazol-5-yl. Representative examples of benzo-fused 5-membered heterocyclyls are benzoxazol-2-yl, benzthiazol-2-yl and benzimidazol-2-yl. Example 6-membered heterocyclyls contain one to three nitrogen atoms and optionally a sulfur or oxygen atom, for example pyridyl, such as pyrid-2-yl, pyrid-3-yl, and pyrid-4-yl; pyrimidyl, such as pyrimid-2-yl and pyrimid-4-yl; triazinyl, such as 1,3,4-triazin-2-yl and 1,3,5-triazin-4-yl; pyridazinyl, in particular pyridazin-3-yl, and pyrazinyl. The pyridine N-oxides and pyridazine N-oxides and the pyridyl, pyrimid-2-yl, pyrimid-4-yl, pyridazinyl and the 1,3,4-triazin-2-yl groups, are yet other examples of heterocyclyl groups. In some embodiments, a heterocyclic group includes a heterocyclic ring fused to one or more (e.g., 1, 2 or 3) different cyclic groups (e.g., carbocyclic rings or heterocyclic rings), where the radical or point of attachment is on the heterocyclic ring, and in some embodiments wherein the point of attachment is a heteroatom contained in the heterocyclic ring.

Thus, the term heterocyclic embraces N-heterocyclyl groups which as used herein refer to a heterocyclyl group containing at least one nitrogen and where the point of attachment of the heterocyclyl group to the rest of the molecule is through a nitrogen atom in the heterocyclyl group. Representative examples of N-heterocyclyl groups include 1-morpholinyl, 1-piperidinyl, 1-piperazinyl, 1-pyrrolidinyl, pyrazolidinyl, imidazolinyl and imidazolidinyl. The term heterocyclic also embraces C-heterocyclyl groups which as used herein refer to a heterocyclyl group containing at least one heteroatom and where the point of attachment of the heterocyclyl group to the rest of the molecule is through a carbon atom in the heterocyclyl group. Representative examples of C-heterocyclyl radicals include 2-morpholinyl, 2- or 3- or 4-piperidinyl, 2-piperazinyl, and 2- or 3-pyrrolidinyl. The term heterocyclic also embraces heterocyclylalkyl groups which as disclosed above refer to a group of the formula —R^{c}-heterocyclyl where RC is an alkylene chain. The term heterocyclic also embraces heterocyclylalkoxy groups which as used herein refer to a radical bonded through an oxygen atom of the formula —O—R^{c}-heterocyclyl where R^{c} is an alkylene chain.

As used herein, the term “heteroaryl” used alone or as part of a larger moiety (e.g., “heteroarylalkyl” (also “heteroaralkyl”), or “heteroarylalkoxy” (also “heteroaralkoxy”), refers to a monocyclic, bicyclic or tricyclic ring system having 5 to 14 ring atoms, wherein at least one ring is aromatic and contains at least one heteroatom. In one embodiment, heteroaryl includes 5-6 membered monocyclic aromatic groups where one or more ring atoms is nitrogen, sulfur or oxygen. Representative examples of heteroaryl groups include thienyl, furyl, imidazolyl, pyrazolyl, thiazolyl, isothiazolyl, oxazolyl, isoxazolyl, triazolyl, thiadiazolyl, oxadiazolyl, tetrazolyl, thiatriazolyl, oxatriazolyl, pyridyl, pyrimidyl, imidazopyridyl, pyrazinyl, pyridazinyl, triazinyl, tetrazinyl, tetrazolo[1,5-b]pyridazinyl, purinyl, deazapurinyl, benzoxazolyl, benzofuryl, benzothiazolyl, benzothiadiazolyl, benzotriazolyl, benzoimidazolyl, indolyl, 1,3-thiazol-2-yl, 1,3,4-triazol-5-yl, 1,3-oxazol-2-yl, 1,3,4-oxadiazol-5-yl, 1,2,4-oxadiazol-5-yl, 1,3,4-thiadiazol-5-yl, 1H-tetrazol-5-yl, 1,2,3-triazol-5-yl, and pyrid-2-yl N-oxide. The term “heteroaryl” also includes groups in which a heteroaryl is fused to one or more cyclic (e.g., carbocyclyl, or heterocyclyl) rings, where the radical or point of attachment is on the heteroaryl ring. Nonlimiting examples include indolyl, indolizinyl, isoindolyl, benzothienyl, benzothiophenyl, methylenedioxyphenyl, benzofuranyl, dibenzofuranyl, indazolyl, benzimidazolyl, benzodioxazolyl, b enzthi az olyl, quinolyl, isoquinolyl, cinnolinyl, phthalazinyl, quinazolinyl, quinoxalinyl, 4H-quinolizinyl, carbazolyl, acridinyl, phenazinyl, phenothiazinyl, phenoxazinyl, tetrahydroquinolinyl, tetrahydroisoquinolinyl and pyrido[2,3-b]-1,4-oxazin-3(4H)-one. A heteroaryl group may be mono-, bi- or tri-cyclic. In some embodiments, a heteroaryl group includes a heteroaryl ring fused to one or more (e.g., 1, 2 or 3) different cyclic groups (e.g., carbocyclic rings or heterocyclic rings), where the radical or point of attachment is on the heteroaryl ring, and in some embodiments wherein the point of attachment is a heteroatom contained in the heterocyclic ring.

Thus, the term heteroaryl embraces N-heteroaryl groups which as used herein refer to a heteroaryl group as defined above containing at least one nitrogen and where the point of attachment of the heteroaryl group to the rest of the molecule is through a nitrogen atom in the heteroaryl group. The term heteroaryl also embraces C-heteroaryl groups which as used herein refer to a heteroaryl group as defined above and where the point of attachment of the heteroaryl group to the rest of the molecule is through a carbon atom in the heteroaryl group. The term heteroaryl also embraces heteroarylalkyl groups which as disclosed above refer to a group of the formula —R^{c}-heteroaryl, wherein R^{c} is an alkylene chain as defined above. The term heteroaryl also embraces heteroaralkoxy (or heteroarylalkoxy) groups which as used herein refer to a group bonded through an oxygen atom of the formula —O—R^{c}-heteroaryl, where R^{c} is an alkylene group as defined above.

As used herein, the term “heterocyclene” refers to a bivalent heterocyclyl radical which may be optionally substituted.

As used herein, the term “heteroarylene” refers to a bivalent heteroaryl radical which may be optionally substituted.

As used herein, the term “amino acid” refers to arginine, histidine, lysine, aspartic acid, glutamic acid, serine, threonine, asparagine, glutamine, cysteine, selenocysteine, glycine, proline, alanine, isoleucine, leucine, methionine, phenylalanine, tryptophan, tyrosine, valine, and modifications thereof. Modifications include phosphorylation, methylation, acetylation, amidation, formation of pyrrolidone carboxylic acid, isomerization, hydroxylation, sulfation, flavin-binding, cysteine oxidation and nitrosylation.

Unless stated otherwise, and to the extent not further defined for any particular group(s), any of the groups described herein may be substituted or unsubstituted. As used herein, the term “substituted” broadly refers to all permissible substituents with the implicit proviso that such substitution is in accordance with permitted valence of the substituted atom and the substituent, and that the substitution results in a stable compound, i.e., a compound that does not spontaneously undergo transformation such as by rearrangement, cyclization, elimination, etc. Representative substituents include halogens, hydroxyl groups, and any other organic groupings containing any number of carbon atoms, e.g., 1-14 carbon atoms, and which may include one or more (e.g., 1, 2, 3, or 4) heteroatoms such as oxygen, sulfur, and nitrogen grouped in a linear, branched, or cyclic structural format.

To the extent not disclosed otherwise for any particular group(s), representative examples of substituents may include alkyl, substituted alkyl (e.g., C₁-C₆, C₁-C₅, C₁-C₄, C₁-C₃, C₁-C₂, C₁), alkoxy (e.g., C₁-C₆, C₁-C₅, C₁-C₄, C₁-C₃, C₁-C₂, C₁), substituted alkoxy (e.g., C₁-C₆, C₁-C₅, C₁-C₄, C₁-C₃, C₁-C₂, C₁), haloalkyl (e.g., CF₃), alkenyl (e.g., C₂-C₆, C₁-C₅, C₁-C₄, C₁-C₃, C₂), substituted alkenyl (e.g., C₂-C₆, C₂-C₅, C₂-C₄, C₂-C₃, C₂), alkynyl (e.g., C₂-C₆, C₂-C₅, C₂-C₄, C₂-C₃, C₂), substituted alkynyl (e.g., C₂-C₆, C₂-C₅, C₂-C₄, C₂-C₃, C₂), cyclic (e.g., C₃-C₁₂, C₅-C₆), substituted cyclic (e.g., C₃-C₁₂, C₅-C₆, ), carbocyclic (e.g., C₃-C₁₂, C₅-C₆), substituted carbocyclic (e.g., C₃-C₁₂, C₅-C₆), heterocyclic (e.g., C₃-C₁₂, C₅-C₆), substituted heterocyclic (e.g., C₃-C₁₂, C₅-C₆), aryl (e.g., benzyl and phenyl), substituted aryl (e.g., substituted benzyl or phenyl), heteroaryl (e.g., pyridyl or pyrimidyl), substituted heteroaryl (e.g., substituted pyridyl or pyrimidyl), aralkyl (e.g., benzyl), substituted aralkyl (e.g., substituted benzyl), halo, hydroxyl, aryloxy (e.g., C₆-C₁₂, C₆), substituted aryloxy (e.g., C₆-C₁₂, C₆), alkylthio (e.g., C₁-C₆), substituted alkylthio (e.g., C₁-C₆), arylthio (e.g., C₆-C₁₂, C₆), substituted arylthio (e.g., C₆-C₁₂, C₆), cyano, carbonyl, substituted carbonyl, carboxyl, substituted carboxyl, amino, substituted amino, amido, substituted amido, thio, substituted thio, sulfinyl, substituted sulfinyl, sulfonyl, substituted sulfonyl, sulfinamide, substituted sulfinamide, sulfonamide, substituted sulfonamide, urea, substituted urea, carbamate, substituted carbamate, amino acid, and peptide groups.

In one aspect, compounds of the disclosure are represented by Formula I:

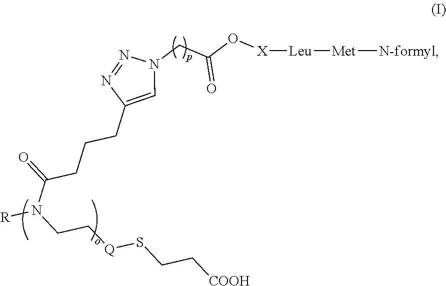

wherein,

o is an integer from 1 to 50;

p is an integer from 1 to 20;

Q is a polyoxazoline polymer;

R is hydrogen, unsubstituted or substituted alkyl, unsubstituted or substituted alkenyl, unsubstituted or substituted aralkyl, or unsubstituted or substituted heterocyclylalkyl; and

X is an amino acid chain between 1-50 amino acids in length wherein at least one of the amino acids is a hydroxyl-containing amino acid, or a pharmaceutically acceptable salt or stereoisomer thereof.

In some embodiments, the polyoxazoline polymer is

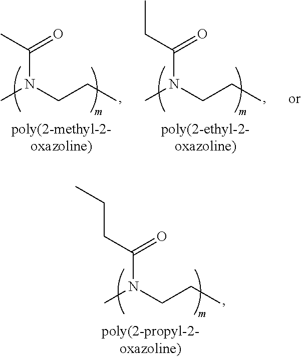

wherein m is an integer from 190 to 950. In some embodiments, the polyoxazoline polymer is

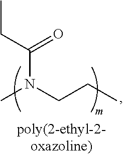

wherein m is an integer from 190 to 950. Bioconjugation to poly(2-ethyl-2-oxazoline) (PEOZ) prolongs blood circulation time and improves drug efficacy due to its high solubility in aqueous media, biocompatibility, and good tolerance. Without being bound by theory, it is believed that PEOZ may reduce or even negate one or more limitations of non-derivatized N-fMLF compounds such as rapid clearance, proteolysis, and immunogenicity, and may also provide a camouflaging shield around the protein and produce an apparent hydrodynamic size that reduces proteolysis and renal glomerular filtration. Other synthetic polymers that can be used in the present disclosure, and in place of PEOZ, include:

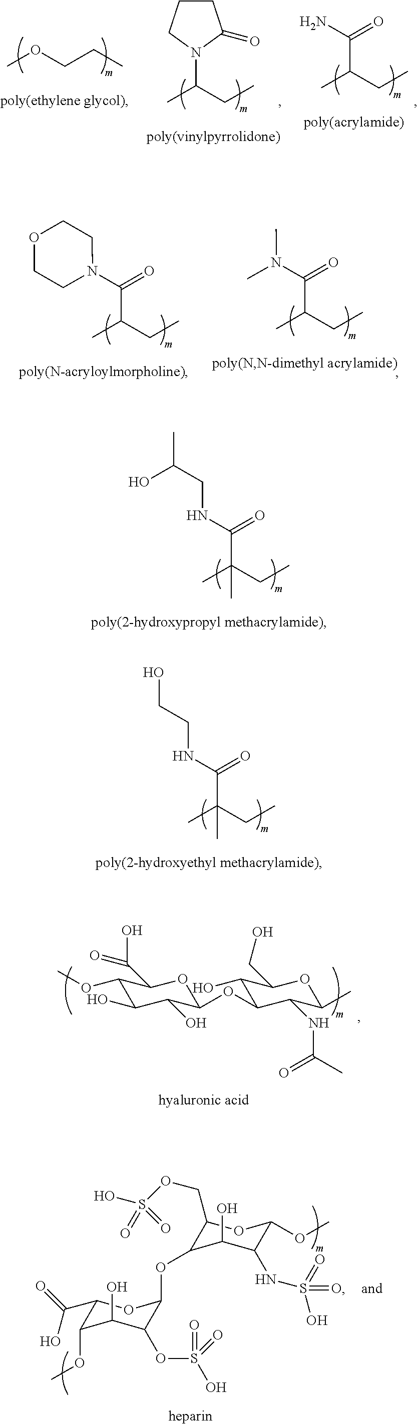

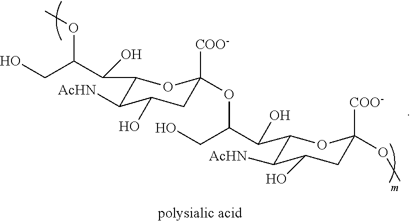

Compounds containing these synthetic polymers are represented by Formula I′:

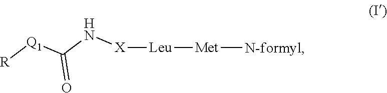

wherein,

Q₁ is a synthetic polymer;

R is hydrogen, unsubstituted or substituted alkyl, unsubstituted or substituted alkenyl, unsubstituted or substituted aralkyl, or unsubstituted or substituted heterocyclylalkyl; and

X is an amino acid chain between 1-50 amino acids in length wherein at least one of the amino acids is a hydroxyl-containing amino acid, or a pharmaceutically acceptable salt or stereoisomer thereof.

In some embodiments, m is an integer from 190 to 350; o is an integer from 1 to 10; p is an integer from 1 to 10; and R is hydrogen, alkyl, or substituted alkyl.

In some embodiments, X is 1-11 amino acids in length. In some embodiments, X is 1-5 amino acids in length. In some embodiments, X is 1-3 amino acids in length.

In some embodiments, m is an integer from 190 to 850. In some embodiments, m is an integer from 190 to 750. In some embodiments, m is an integer from 190 to 650. In some embodiments, m is an integer from 190 to 550. In some embodiments, m is an integer from 190 to 450. In some embodiments, m is an integer from 190 to 350.

In some embodiments, o is an integer from 1 to 40. In some embodiments, o is an integer from 1 to 30. In some embodiments, o is an integer from 1 to 20. In some embodiments, o is an integer from 1 to 10. In some embodiments, o is an integer from 1 to 5.

In some embodiments, p is an integer from 1 to 15. In some embodiments, p is an integer from 1 to 10. In some embodiments, p is an integer from 1 to 5.

In some embodiments, R is hydrogen. In some embodiments, R is alkyl. In some embodiments, R is methyl.

In some embodiments, X is selected from Tyr, Tyr-Phe, Phe-Tyr, Tyr-Phe-Phe, Phe-Phe-Tyr, Phe-Tyr-Phe, Ser, Ser-Phe, Phe-Ser, Ser-Phe-Phe, Phe-Phe-Ser, Phe-Ser-Phe, Thr, Thr-Phe, Phe-Thr, Thr-Phe-Phe, Phe-Phe-Thr, Phe-Thr-Phe, and Tyr-Tyr-Tyr.

In some embodiments, X is Tyr-Phe (Formula I-a), Phe-Tyr (Formula I-b), Tyr-Tyr (Formula I-c), Phe-Phe-Tyr (Formula I-d), Phe-Tyr-Phe (Formula I-e), Tyr-Phe-Phe (Formula I-f), or Tyr-Tyr-Tyr (Formula I-g).

In some embodiments, the compounds may be represented by any of formulas I-a to I-g, as follows:

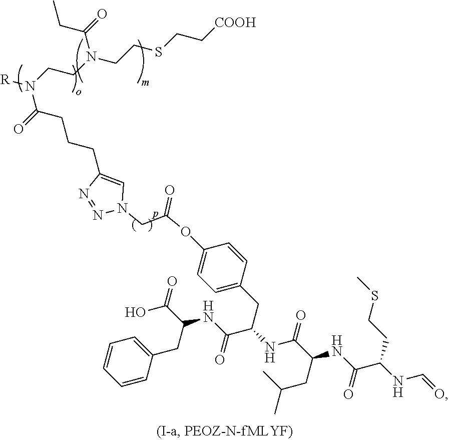

wherein m is an integer from 5 to 20; o is an integer from 1 to 4;
p is an integer from 1 to 5; and R is methyl,

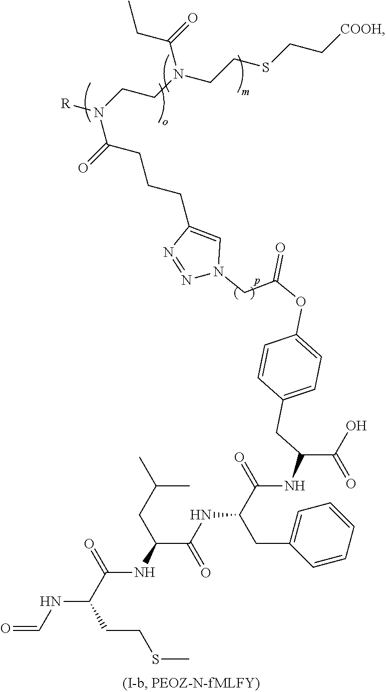

wherein m is an integer from 5 to 20; o is an integer from 1 to 4;
p is an integer from 1 to 5; and R is methyl,

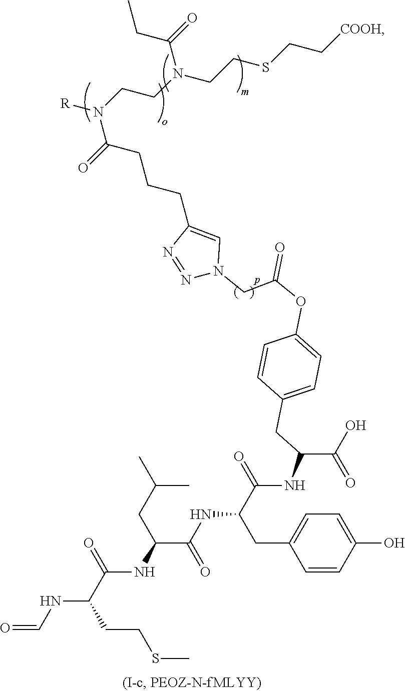

wherein m is an integer from 5 to 20; o is an integer from 1 to 4;
p is an integer from 1 to 5; and R is methyl,

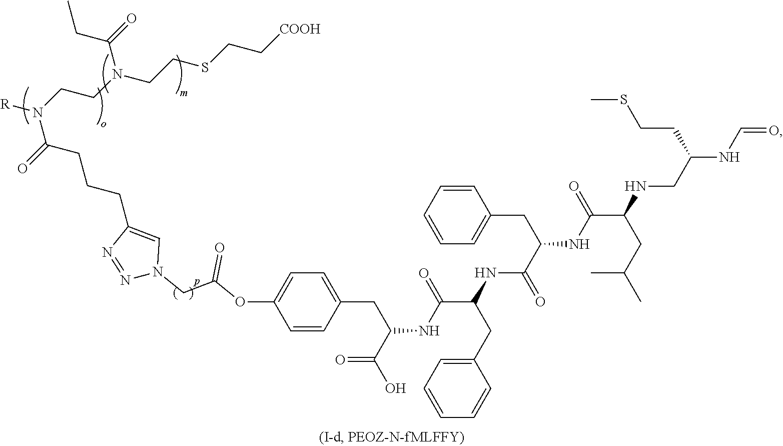

wherein m is an integer from 5 to 20; o is an integer from 1 to 4;
p is an integer from 1 to 5; and R is methyl,

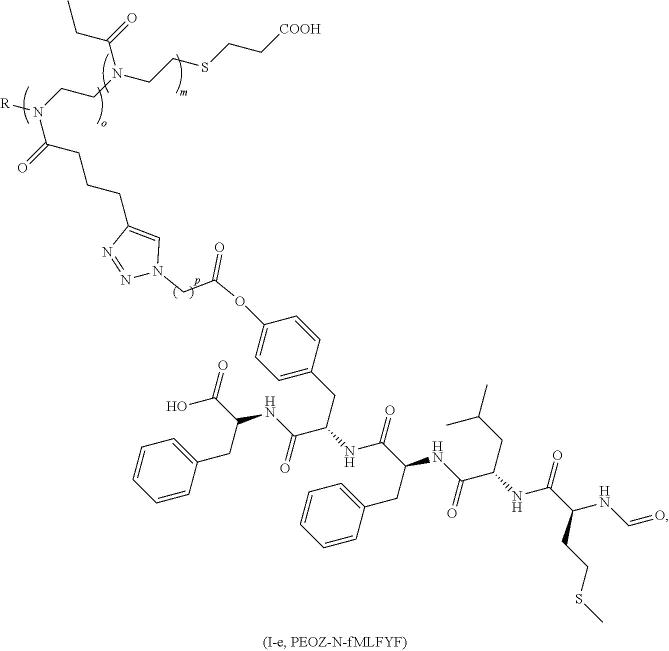

wherein m is an integer from 5 to 20; o is an integer from 1 to 4;
p is an integer from 1 to 5; and R is methyl,

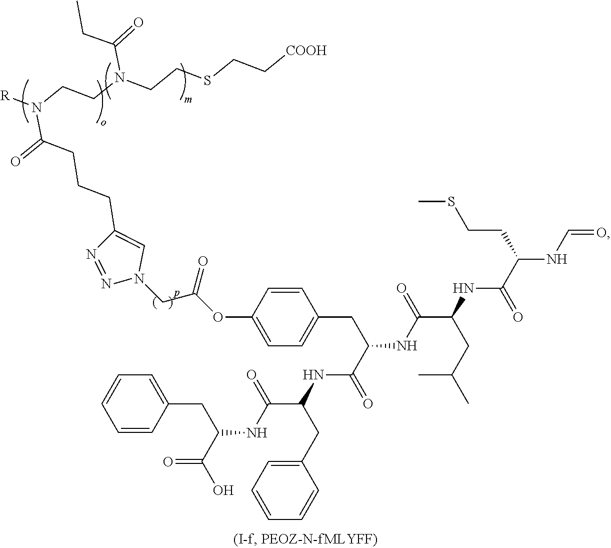

wherein m is an integer from 5 to 20; o is an integer from 1 to 4;
p is an integer from 1 to 5; and R is methyl, and

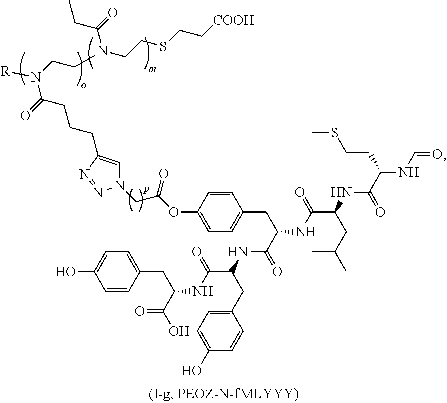

wherein m is an integer from 5 to 20; o is an integer from 1 to 4;
p is an integer from 1 to 5; and R is methyl.

Compounds of the present disclosure may be in the form of a free acid or free base, or a pharmaceutically acceptable salt. As used herein, the term “pharmaceutically acceptable” in the context of a salt refers to a salt of the compound that does not abrogate the biological activity or properties of the compound, and is relatively non-toxic, i.e., the compound in salt form may be administered to a subject without causing undesirable biological effects (such as dizziness or gastric upset) or interacting in a deleterious manner with any of the other components of the composition in which it is contained. The term “pharmaceutically acceptable salt” refers to a product obtained by reaction of the compound of the present disclosure with a suitable acid or a base. Examples of pharmaceutically acceptable salts of the compounds of this disclosure include those derived from suitable inorganic bases such as Li, Na, K, Ca, Mg, Fe, Cu, Al, Zn and Mn salts. Examples of pharmaceutically acceptable, nontoxic acid addition salts are salts of an amino group formed with inorganic acids such as hydrochloride, hydrobromide, hydroiodide, nitrate, sulfate, bisulfate, phosphate, isonicotinate, acetate, lactate, salicylate, citrate, tartrate, pantothenate, bitartrate, ascorbate, succinate, maleate, gentisate, fumarate, gluconate, glucoronate, saccharate, formate, benzoate, glutamate, methanesulfonate, ethanesulfonate, benzenesulfonate, 4-methylbenzenesulfonate or p-toluenesulfonate salts and the like. Certain compounds of the disclosure can form pharmaceutically acceptable salts with various organic bases such as lysine, arginine, guanidine, diethanolamine or metformin. Suitable base salts include aluminum, calcium, lithium, magnesium, potassium, sodium, or zinc salts. In some embodiments, pharmaceutically acceptable salts of the compounds of this disclosure are potassium salts.

Compounds of the present disclosure may have at least one chiral center and thus may be in the form of a stereoisomer, which as used herein, embraces all isomers of individual compounds that differ only in the orientation of their atoms in space. The term stereoisomer includes mirror image isomers (enantiomers which include the (R-) or (S-) configurations of the compounds), mixtures of mirror image isomers (physical mixtures of the enantiomers, and racemates or racemic mixtures) of compounds, geometric (cis/trans or E/Z, R/S) isomers of compounds and isomers of compounds with more than one chiral center that are not mirror images of one another (diastereoisomers). The chiral centers of the compounds may undergo epimerization in vivo; thus, for these compounds, administration of the compound in its (R-) form is considered equivalent to administration of the compound in its (S-) form. Accordingly, the compounds of the present disclosure may be made and used in the form of individual isomers and substantially free of other isomers, or in the form of a mixture of various isomers, e.g., racemic mixtures of stereoisomers.

In some embodiments, the compound is an isotopic derivative in that it has at least one desired isotopic substitution of an atom, at an amount above the natural abundance of the isotope, i.e., enriched. In one embodiment, the compound includes deuterium or multiple deuterium atoms. In one embodiment, the compound includes one or more heavy or light atoms of C, P, N, and S. Substitution with heavier isotopes such as deuterium, i.e. ²H, may afford certain therapeutic advantages resulting from greater metabolic stability, for example, increased in vivo half-life or reduced dosage requirements, and thus may be advantageous in some circumstances.

The compounds of the present disclosure may be prepared by crystallization under different conditions and may exist as one or a combination of polymorphs of the compound. For example, different polymorphs may be identified and/or prepared using different solvents, or different mixtures of solvents for recrystallization, by performing crystallizations at different temperatures, or by using various modes of cooling, ranging from very fast to very slow cooling during crystallizations. Polymorphs may also be obtained by heating or melting the compound followed by gradual or fast cooling. The presence of polymorphs may be determined by solid probe NMR spectroscopy, IR spectroscopy, differential scanning calorimetry, powder X-ray diffractogram and/or other known techniques.

### Pharmaceutical Compositions

Another aspect of the present disclosure is directed to a pharmaceutical composition that includes a therapeutically effective amount of a compound of Formula (I or I′) or a pharmaceutically acceptable salt or stereoisomer thereof, and a pharmaceutically acceptable carrier. The term “pharmaceutically acceptable carrier,” as known in the art, refers to a pharmaceutically acceptable material, composition or vehicle, suitable for administering compounds of the present disclosure to mammals. Suitable carriers may include, for example, liquids (both aqueous and non-aqueous alike, and combinations thereof), solids, encapsulating materials, gases, and combinations thereof (e.g., semi-solids), and gases, that function to carry or transport the compound from one organ, or portion of the body, to another organ, or portion of the body. A carrier is “acceptable” in the sense of being physiologically inert to and compatible with the other ingredients of the formulation and not injurious to the subject or patient. Depending on the type of formulation, the composition may also include one or more pharmaceutically acceptable excipients.

Broadly, compounds of Formula (I or I′) and their pharmaceutically acceptable salts and stereoisomers may be formulated into a given type of composition in accordance with conventional pharmaceutical practice such as conventional mixing, dissolving, granulating, dragee-making, levigating, emulsifying, encapsulating, entrapping and compression processes (see, e.g., Remington: The Science and Practice of Pharmacy (20th ed.), ed. A. R. Gennaro, Lippincott Williams & Wilkins, 2000 and Encyclopedia of Pharmaceutical Technology, eds. J. Swarbrick and J. C. Boylan, 1988-1999, Marcel Dekker, New York). The type of formulation depends on the mode of administration which may include enteral (e.g., oral, buccal, sublingual and rectal), parenteral (e.g., subcutaneous (s.c.), intravenous (i. v.), intramuscular (i.m.), and intrasternal injection, or infusion techniques, intra-ocular, intra-arterial, intramedullary, intrathecal, intraventricular, transdermal, interdermal, intravaginal, intraperitoneal, mucosal, nasal, intratracheal instillation, bronchial instillation, and inhalation) and topical (e.g., transdermal). In general, the most appropriate route of administration will depend upon a variety of factors including, for example, the nature of the agent (e.g., its stability in the environment of the gastrointestinal tract), and/or the condition of the subject (e.g., whether the subject is able to tolerate oral administration). For example, parenteral (e.g., intravenous) administration may also be advantageous in that the compound may be administered relatively quickly such as in the case of a single-dose treatment and/or an acute condition.

In some embodiments, the compounds are formulated for oral or intravenous administration (e.g., systemic intravenous injection).

Accordingly, compounds of Formula (I or I′) may be formulated into solid compositions (e.g., powders, tablets, dispersible granules, capsules, cachets, and suppositories), liquid compositions (e.g., solutions in which the compound is dissolved, suspensions in which solid particles of the compound are dispersed, emulsions, and solutions containing liposomes, micelles, or nanoparticles, syrups and elixirs); semi-solid compositions (e.g., gels, suspensions and creams); and gases (e.g., propellants for aerosol compositions). Compounds may also be formulated for rapid, intermediate or extended release.

Injectable preparations for parenteral administration may include sterile aqueous solutions or oleaginous suspensions. They may be formulated according to standard techniques using suitable dispersing or wetting agents and suspending agents. The sterile injectable preparation may also be a sterile injectable solution, suspension or emulsion in a nontoxic parenterally acceptable diluent or solvent, for example, as a solution in 1,3-butanediol dimethylsulfoxide (DMSO). Among the acceptable vehicles and solvents that may be employed are water, Ringer's solution, U.S.P. and isotonic sodium chloride solution. In addition, sterile, fixed oils are conventionally employed as a solvent or suspending medium. For this purpose any bland fixed oil can be employed including synthetic mono- or diglycerides. In addition, fatty acids such as oleic acid are used in the preparation of injectables. The injectable formulations can be sterilized, for example, by filtration through a bacterial-retaining filter, or by incorporating sterilizing agents in the form of sterile solid compositions which can be dissolved or dispersed in sterile water or other sterile injectable medium prior to use. The effect of the compound may be prolonged by slowing its absorption, which may be accomplished by the use of a liquid suspension or crystalline or amorphous material with poor water solubility. Prolonged absorption of the compound from a parenterally administered formulation may also be accomplished by suspending the compound in an oily vehicle.

In certain embodiments, compounds of Formula (I or I′) may be administered in a local rather than systemic manner, for example, via injection of the conjugate directly into an organ, often in a depot preparation or sustained release formulation. In specific embodiments, long-acting formulations are administered by implantation (for example subcutaneously or intramuscularly) or by intramuscular injection. Injectable depot forms are made by forming microencapsule matrices of the compound in a biodegradable polymer, e.g., polylactide-polyglycolides, poly(orthoesters) and poly(anhydrides). The rate of release of the compound may be controlled by varying the ratio of compound to polymer and the nature of the particular polymer employed. Depot injectable formulations are also prepared by entrapping the compound in liposomes or microemulsions that are compatible with body tissues. Furthermore, in other embodiments, the compound is delivered in a targeted drug delivery system, for example, in a liposome coated with organ-specific antibody. In such embodiments, the liposomes are targeted to and taken up selectively by the organ.

The compounds of Formula (I or I′) may be formulated for administration by inhalation. Various forms suitable for administration by inhalation include aerosols, mists or powders. Pharmaceutical compositions may be delivered in the form of an aerosol spray presentation from pressurized packs or a nebulizer, with the use of a suitable propellant (e.g., dichlorodifluoromethane, trichlorofluoromethane, dichlorotetrafluoroethane, carbon dioxide or other suitable gas). In some embodiments, the dosage unit of a pressurized aerosol may be determined by providing a valve to deliver a metered amount. In some embodiments, capsules and cartridges including gelatin, for example, for use in an inhaler or insufflator, may be formulated containing a powder mix of the compound and a suitable powder base such as lactose or starch.

Compounds of Formula (I or I′) may be formulated for topical administration which as used herein, refers to administration intradermally by disclosure of the formulation to the epidermis. These types of compositions are typically in the form of ointments, pastes, creams, foams, lotions, gels, solutions and sprays.

Representative examples of carriers useful in formulating compounds for topical application include solvents (e.g., alcohols, poly alcohols, water), creams, lotions, ointments, oils, plasters, liposomes, powders, emulsions, microemulsions, and buffered solutions (e.g., hypotonic or buffered saline). Creams, for example, may be formulated using saturated or unsaturated fatty acids such as stearic acid, palmitic acid, oleic acid, palmito-oleic acid, cetyl, or oleyl alcohols. Creams may also contain a non-ionic surfactant such as polyoxy-40-stearate.

In some embodiments, the topical formulations may also include an excipient, an example of which is a penetration enhancing agent. These agents are capable of transporting a pharmacologically active compound through the stratum corneum and into the epidermis or dermis, preferably, with little or no systemic absorption. A wide variety of compounds have been evaluated as to their effectiveness in enhancing the rate of penetration of drugs through the skin. See, for example, Percutaneous Penetration Enhancers, Maibach H. I. and Smith H. E. (eds.), CRC Press, Inc., Boca Raton, Fla. (1995), which surveys the use and testing of various skin penetration enhancers, and Buyuktimkin et al., Chemical Means of Transdermal Drug Permeation Enhancement in Transdermal and Topical Drug Delivery Systems, Gosh T. K., Pfister W. R., Yum S. I. (Eds.), Interpharm Press Inc., Buffalo Grove, Ill. (1997). Representative examples of penetration enhancing agents include triglycerides (e.g., soybean oil), aloe compositions (e.g., aloe-vera gel), ethyl alcohol, isopropyl alcohol, octolyphenylpolyethylene glycol, oleic acid, polyethylene glycol 400, propylene glycol, N-decylmethylsulfoxide, fatty acid esters (e.g., isopropyl myristate, methyl laurate, glycerol monooleate, and propylene glycol monooleate), and N-methylpyrrolidone.

Representative examples of yet other excipients that may be included in topical as well as in other types of formulations (to the extent they are compatible), include preservatives, antioxidants, moisturizers, emollients, buffering agents, solubilizing agents, skin protectants, and surfactants. Suitable preservatives include alcohols, quaternary amines, organic acids, parabens, and phenols. Suitable antioxidants include ascorbic acid and its esters, sodium bisulfite, butylated hydroxytoluene, butylated hydroxyanisole, tocopherols, and chelating agents like EDTA and citric acid. Suitable moisturizers include glycerin, sorbitol, polyethylene glycols, urea, and propylene glycol. Suitable buffering agents include citric, hydrochloric, and lactic acid buffers. Suitable solubilizing agents include quaternary ammonium chlorides, cyclodextrins, benzyl benzoate, lecithin, and polysorbates. Suitable skin protectants include vitamin E oil, allatoin, dimethicone, glycerin, petrolatum, and zinc oxide.

In some embodiments, the topical formulation further comprises a non-steroidal anti-inflammatory drug (NSAID). Representative examples of NSAIDs include acetylsalicylic acid (aspirin), dolobid (diflunisal), salicyclic acid, disalcid (salsalate), ibuprofen, dexibuprofen, naproxen, fenoprofen, ketoprofen, dexketoprofen, flubiprofen, oxaprozin, loxoprofen, pelubiprofen, zaltoprofen, indomethacin, tolmetin, sulindac, diclofenac, aceclofenac, bromfenac, etodolac, ketorolac, nabumetone, piroxicam, meloxicam, tenoxicam, droxicam, lornoxicam, isoxicam, phenylbutazone, mefenamic acid, meclofenamic acid, flufenamic acid, tolfenamic acid, and clonixin.

In some embodiments, the topical formulation further comprises an analgesic. Representative analgesics include paracetamol (acetaminophen), COX-2 inhibitors (e.g., rofecoxib, celecoxib, and etoricoxib), opioids (e.g., morphine, codeine, oxycodone, hydrocodone, dihydromorphine, pethidine, buprenorphine, tramadol, venlafaxine, tapentadol, serotonin, and norepinephrine), nefopam, flupiritine, and ziconotide.

Transdermal formulations typically employ transdermal delivery devices and transdermal delivery patches wherein the compound is formulated in lipophilic emulsions or buffered, aqueous solutions, dissolved and/or dispersed in a polymer or an adhesive. Patches may be constructed for continuous, pulsatile, or on demand delivery of pharmaceutical agents. Transdermal delivery of the compounds may be accomplished by means of an iontophoretic patch. Transdermal patches may provide controlled delivery of the compounds wherein the rate of absorption is slowed by using rate-controlling membranes or by trapping the compound within a polymer matrix or gel. Absorption enhancers may be used to increase absorption, examples of which include absorbable pharmaceutically acceptable solvents that assist passage through the skin.

### Methods of Use

In some aspects, the present disclosure is directed to treating diseases or disorders characterized or mediated by FPR1 or FPR2. The methods entail administering a therapeutically effective amount of a compound of Formula (I or I′) or a pharmaceutically acceptable salt or stereoisomer thereof, to a subject in need thereof.

A “disease” is generally regarded as a state of health of a subject wherein the subject cannot maintain homeostasis, and wherein if the disease is not ameliorated then the subject's health continues to deteriorate. In contrast, a “disorder” in a subject is a state of health in which the subject is able to maintain homeostasis, but in which the subject's state of health is less favorable than it would be in the absence of the disorder. Left untreated, a disorder does not necessarily cause a further decrease in the subject's state of health.

The term “subject” (or “patient”) as used herein includes all members of the animal kingdom prone to or suffering from the indicated disease or disorder. In some embodiments, the subject is a mammal, e.g., a human or a non-human mammal. The methods are also applicable to companion animals such as dogs and cats as well as livestock such as cows, horses, sheep, goats, pigs, and other domesticated and wild animals. A subject “in need of” treatment according to the present disclosure may be “suffering from or suspected of suffering from” a specific disease or disorder may have been positively diagnosed or otherwise presents with a sufficient number of risk factors or a sufficient number or combination of signs or symptoms such that a medical professional could diagnose or suspect that the subject was suffering from the disease or disorder. Thus, subjects suffering from a specific disease or disorder, and subjects suspected of suffering from a specific disease or disorder are not necessarily two distinct groups.

As used herein, the term, “therapeutically effective amount” refers to an amount of a compound of Formula (I or I′) or a pharmaceutically acceptable salt or a stereoisomer thereof that is effective in producing the desired therapeutic response in a particular patient suffering from a disease or disorder mediated by FPR1 or FPR2 activity. The term “therapeutically effective amount” thus includes the amount of the compound or a pharmaceutically acceptable salt or a stereoisomer thereof, that when administered, induces a positive modification in the disease or disorder to be treated, or is sufficient to prevent development or progression of the disease or disorder, or alleviate to some extent, one or more of the symptoms of the disease or disorder being treated in a subject, or which simply kills or inhibits the growth of diseased cells, or reduces the amounts of FPR1 or FPR2 in diseased cells.

The total daily dosage of the compounds and usage thereof may be decided in accordance with standard medical practice, e.g., by the attending physician using sound medical judgment. The specific therapeutically effective dose for any particular subject may depend upon a variety of factors including the disease or disorder being treated and the severity thereof (e.g., its present status); the age, body weight, general health, sex and diet of the subject; the time of administration, route of administration, and rate of excretion of the specific compound employed; the duration of the treatment; drugs used in combination or coincidental with the compound; and like factors well known in the medical arts (see, for example, Goodman and Gilman's, The Pharmacological Basis of Therapeutics, 10th Edition, A. Gilman, J. Hardman and L. Limbird, eds., McGraw-Hill Press, 155-173, 2001).

Compounds of Formula (I or I′) and their pharmaceutically acceptable salts and stereoisomers may be effective over a wide dosage range. In some embodiments, the total daily dosage (e.g., for adult humans) may be at least about 0.0001 mg/kg, 0.0005 mg/kg, 0.001 mg/kg, mg/kg, 0.01 mg/kg, or 0.05 mg/kg, and may range up to about 1.0 mg/kg, 2.0 mg/kg, 4.0 mg/kg, 8.0 mg/kg, 16.0 mg/kg, 32.0 mg/kg, 64.0 mg/kg or higher per day. In some embodiments, the dose may range from about 0.15 to about 0.25 mg/kg. In some embodiments, the dose may be about 0.20 mg/kg.

In some embodiments, the disease or disorder is an inflammatory disease or disorder. Representative examples of the inflammatory diseases or disorders that may be amenable to treatment with the disclosed compounds include inflammatory bowel disease, ulcerative colitis, crohn's disease, nephritis, acute nephritis, chronic nephritis, glomerulonephritis, iga nephropathy, diabetic nephropathy, membranous nephropathy, hydronephrosis, contrast agent nephropathy, pyelonephritis, kidney failure, acute nephritis, chronic nephritis, interstitial nephritis, renal disorder, nephrotic syndrome, hypertensive nephrosclerosis, diabetic glomerulosclerosis, kidney stone, amyloid kidney, renal vein thrombosis, alport syndrome, hepatitis, cirrhosis of the liver, pancreatitis, pneumonia, sinusitis, psoriasis, rhinitis, arthritis, rheumatoid arthritis, chronic obstructive pulmonary disease (COPD), periodic fever aphthous stomatitis pharyngitis lymphadenitis syndrome (PFAPA), adult onset still's disease, behcet's disease, gout, pseudogout, schnitzler syndrome, chronic recurrent multiple osteomyelitis (CRMO), cryopyrine related periodic fever syndrome (CAPS), familial cold urticaria, muckle-wells syndrome, chronic infantile neuroderm arthritis syndrome (CINCA syndrome)/neonatal onset multiple organ inflammatory disease (NOMID), tumor necrosis factor (TNF) receptor related periodic syndrome (TRAPS), High IgD syndrome (mevalonate kinase deficiency), Blau syndrome/juvenile onset sarcoidosis, familial Mediterranean fever, purulent arthritis/pyoderma gangrenosum/acne syndrome (PAPA), Nakajo-Nishimura syndrome, majeed syndrome, NLRP 12 related periodic fever syndrome (NAPS 12), Interleukin 1 receptor antagonist deficiency (DIRA), Interleukin 36 receptor antagonist deficiency (DITRA), phospholipase C.gamma.2-related antibody deficiency/immune disorder (PLAID), HOIL-1 deficiency, SLC29A3 deficiency, CARD 14 abnormality disease, adenosine deaminase 2 (ADA2) deficiency, STING-associated vasculopathy with onset in infancy (SAVI), and NLRC4 abnormality.

In some embodiments, the inflammatory disease or order is characterized by mucosal inflammation.

In some embodiments, the inflammatory disease or disorder is a respiratory disease or disorder.

In some embodiments, the inflammatory disease or disorder is asthma.

In some embodiments, the inflammatory disease or disorder is chronic obstructive pulmonary disease (COPD).

In some embodiments, the inflammatory disease or disorder is eczema.

In some embodiments, the inflammatory disease or disorder is acute lung injury.

In some embodiments, the inflammatory disease or disorder is an intestinal inflammatory disease or disorder.

In some embodiments, the intestinal inflammatory disease or disorder is colitis.

In some embodiments, the inflammatory disease or disorder is arthritis.

In some embodiments, the inflammatory disease or disorder is psoriasis.

In some embodiments, the disease or disorder is a hyper-IgE syndrome (HIES).

In some embodiments, the HIES is autosomal dominant hyper-IgE syndrome (AD-HIES).

In some embodiments, the disease or disorder is irritable bowl disease.

The methods of the present disclosure may entail administration of a compound of formula (I or I′) or a pharmaceutical composition thereof to the patient in a single dose or in multiple doses (e.g., 1, 2, 3, 4, 5, 6, 7, 8, 10, 15, 20, or more doses). For example, the frequency of administration may range from once a day up to about once every eight weeks. In some embodiments, the the compound may be dosed once a day for 1, 2, 3, 4, 5, or 6 weeks. In some embodiments, the compound may be dosed twice a day for 1, 2, 3, 4, 5, or 6 weeks.

### Pharmaceutical Kits

The present compositions may be assembled into kits or pharmaceutical systems. Kits or pharmaceutical systems according to this aspect of the disclosure include a carrier or package such as a box, carton, tube or the like, having in close confinement therein one or more containers, such as vials, tubes, ampoules, or bottles, which contain a compound of the present disclosure or a pharmaceutical composition which contains the compound and a pharmaceutically acceptable carrier wherein the compound and the carrier may be disposed in the same or separate containers. The kits or pharmaceutical systems of the disclosure may also include printed instructions for using the compounds and compositions.

All patent publications and non-patent publications are indicative of the level of skill of those skilled in the art to which this disclosure pertains. All these publications are herein incorporated by reference to the same extent as if each individual publication were specifically and individually indicated as being incorporated by reference.

Although the disclosure herein has been described with reference to particular embodiments, it is to be understood that these embodiments are merely illustrative of the principles and applications of the present disclosure. It is therefore to be understood that numerous modifications may be made to the illustrative embodiments and that other arrangements may be devised without departing from the spirit and scope of the present disclosure as defined by the appended claims.

What is claimed is:

1. A compound of Formula (I): 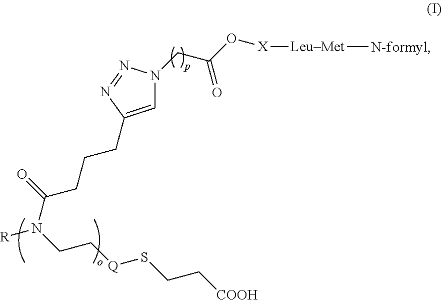
or a stereoisomer or pharmaceutically acceptable salt thereof,
wherein,
o is an integer from 1 to 50;
p is an integer from 1 to 20;
Q is a polyoxazoline polymer;
R is hydrogen, unsubstituted or substituted alkyl, unsubstituted or substituted alkenyl, unsubstituted or substituted aralkyl, or unsubstituted or substituted heterocyclylalkyl; and
X is an amino acid chain between 1-50 amino acids in length wherein at least one of the amino acids is a hydroxyl-containing amino acid.

2. The compound of claim 1, wherein Q is 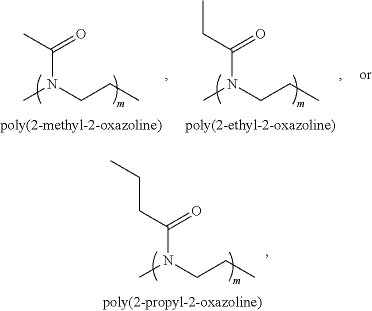
and m is an integer from 190 to 950.

3. The compound of claim 2, wherein Q is 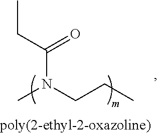
and m is an integer from 190 to 950.

4. The compound of claim 1, wherein:
m is an integer from 190 to 350;
o is an integer from 1 to 10;
p is an integer from 1 to 10; and
R is hydrogen, alkyl, or substituted alkyl.

5. The compound of claim 1, wherein X is 1-11 amino acids in length.

6. The compound of claim 1, wherein X is 1-5 amino acids in length.

7. The compound of claim 1, wherein X is 1-3 amino acids in length.

8. The compound of claim 7, wherein X is selected from the group comprising of:
Tyr, Tyr-Phe, Phe-Tyr, Tyr-Phe-Phe, Phe-Phe-Tyr, Phe-Tyr-Phe, Ser, Ser-Phe, Phe-Ser, Ser-Phe-Phe, Phe-Phe-Ser, Phe-Ser-Phe, Thr, Thr-Phe, Phe-Thr, Thr-Phe-Phe, Phe-Phe-Thr, Phe-Thr-Phe, and Tyr-Tyr-Tyr.

9. The compound of claim 8, wherein X is Tyr-Phe, Phe-Tyr, or Tyr-Tyr.

10. The compound of claim 8, wherein X is Tyr-Tyr-Tyr.

11. The compound of claim 1, which is 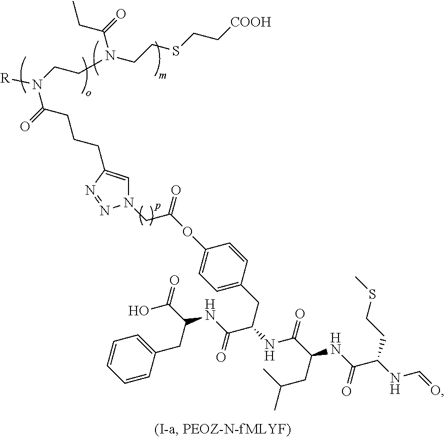
wherein m is an integer from 5 to 20; o is an integer from 1 to 4;
p is an integer from 1 to 5; and R is methyl, 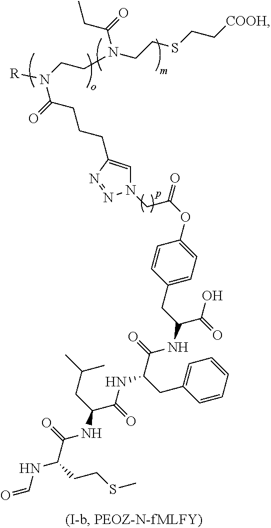
wherein m is an integer from 5 to 20; o is an integer from 1 to 4;
p is an integer from 1 to 5; and R is methyl, 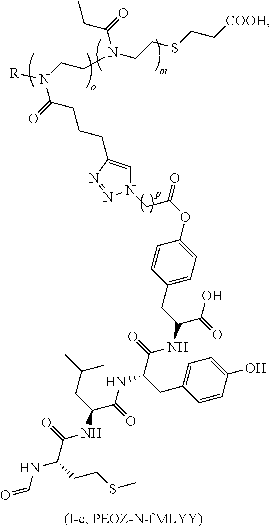
wherein m is an integer from 5 to 20; o is an integer from 1 to 4;
p is an integer from 1 to 5; and R is methyl, or 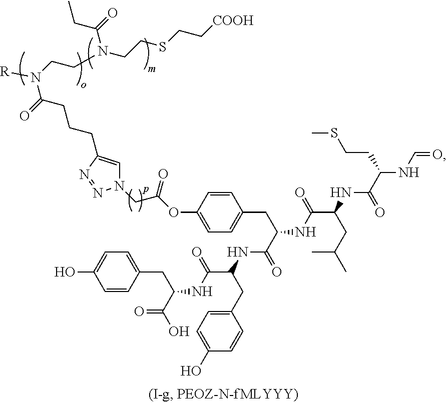
wherein m is an integer from 5 to 20; o is an integer from 1 to 4;
p is an integer from 1 to 5; and R is methyl.

12. A pharmaceutical composition, comprising a therapeutically effective amount of the compound or pharmaceutically acceptable salt or stereoisomer thereof of claim 1, and a pharmaceutically acceptable carrier.

13. The pharmaceutical composition of claim 12, which is in the form of an inhalant and the carrier comprises a propellant.

14. The pharmaceutical composition of claim 12, which is in the form of an injectable formulation.

15. The pharmaceutical composition of claim 14, wherein the carrier comprises an aqueous vehicle.

16. The pharmaceutical composition of claim 12, which is in the form of a topical formulation.

17. The pharmaceutical composition of claim 16, wherein the topical formulation is a cream, ointment, gel, or foam.

18. The pharmaceutical composition of claim 16, further comprising a non-steroidal anti-inflammatory drug (NSAID).

19. The pharmaceutical composition of claim 16, further comprising an analgesic excipient.

20. A method of treating a disease or disorder mediated by Formyl Peptide Receptor 1 or 2, comprising administering to a subject in need thereof a therapeutically effective amount of a compound of Formula (I): 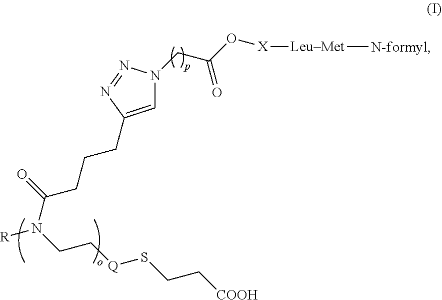
or a stereoisomer or pharmaceutically acceptable salt thereof,
wherein,
o is an integer from 1 to 50;
p is an integer from 1 to 20;
Q is a polyoxazoline polymer;
R is hydrogen, unsubstituted or substituted alkyl, unsubstituted or substituted alkenyl, unsubstituted or substituted aralkyl, or unsubstituted or substituted heterocyclylalkyl; and
X is an amino acid chain between 1-50 amino acids in length wherein at least one of the amino acids is a hydroxyl-containing amino acid.

21. The method of claim 20, wherein the disease or disorder is an inflammatory disease or disorder.

22. The method of claim 21, wherein the inflammatory disease or order is characterized by mucosal inflammation, or wherein the inflammatory disease or disorder is a respiratory disease or disorder, asthma, chronic obstructive pulmonary disease (COPD), eczema, acute lung injury, arthritis, psoriasis, or an intestinal inflammatory disease or disorder.

23. The method of claim 22, wherein the intestinal inflammatory disease or disorder is colitis.

24. The method of claim 20, wherein the disease or disorder is a hyper-IgE syndrome (HIES).

25. The method of claim 24, wherein the HIES is autosomal dominant hyper-IgE syndrome (AD-HIES).

26. The method of claim 20, wherein Q is 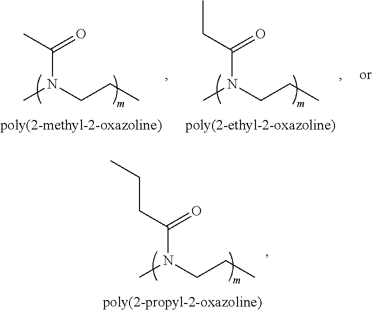
wherein m is an integer from 190 to 950.

27. The method of claim 26, wherein Q is 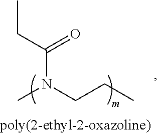
wherein m is an integer from 190 to 950.

28. The method of claim 20, wherein:
m is an integer from 190 to 350;
o is an integer from 1 to 10;
p is an integer from 1 to 10; and
R is hydrogen, alkyl, or substituted alkyl.

29. The method of claim 20, wherein X is 1-11 amino acids in length.

30. The method of claim 20, wherein X is 1-5 amino acids in length.

31. The method of claim 20, wherein X is 1-3 amino acids in length.

32. The method of claim 31, wherein X is selected from the group comprising of:
Tyr, Tyr-Phe, Phe-Tyr, Tyr-Phe-Phe, Phe-Phe-Tyr, Phe-Tyr-Phe, Ser, Ser-Phe, Phe-Ser, Ser-Phe-Phe, Phe-Phe-Ser, Phe-Ser-Phe, Thr, Thr-Phe, Phe-Thr, Thr-Phe-Phe, Phe-Phe-Thr, Phe-Thr-Phe, and Tyr-Tyr-Tyr.

33. The method of claim 32, wherein X is Tyr-Phe, Phe-Tyr or Tyr-Tyr.

34. The method of claim 32, wherein X is Tyr-Tyr-Tyr.

35. The method of claim 20, wherein the compound is 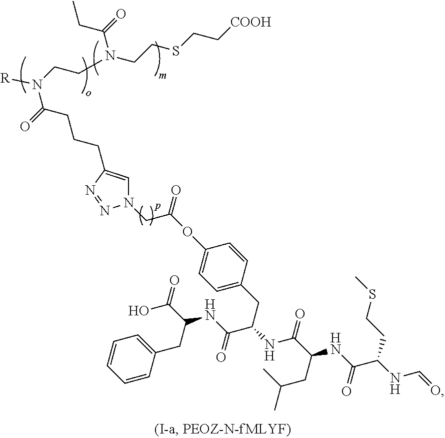
wherein m is an integer from 5 to 20; o is an integer from 1 to 4;
p is an integer from 1 to 5; and R is methyl, 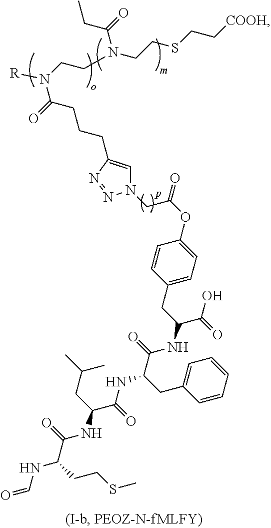
wherein m is an integer from 5 to 20; o is an integer from 1 to 4;
p is an integer from 1 to 5; and R is methyl, 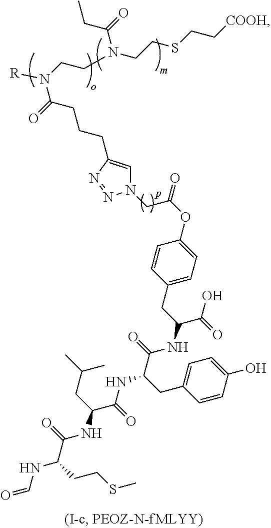
wherein m is an integer from 5 to 20; o is an integer from 1 to 4;
p is an integer from 1 to 5; and R is methyl, or 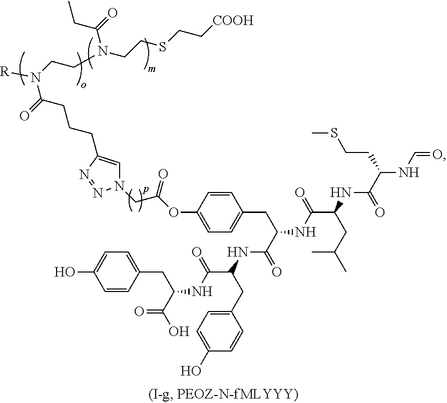
wherein m is an integer from 5 to 20; o is an integer from 1 to 4;
p is an integer from 1 to 5; and R is methyl.
